(19) United States
(12) Patent Application Publication
(10) Pub. No.: US 2016/0151565 A1
(43) Pub. Date: Jun. 2, 2016

(54) FLUID CONTAINER DISPLACEMENT PUMP

(71) Applicant: BAYER HEALTHCARE LLC, Whippany, NJ (US)

(72) Inventors: Kevin P. Cowan, Allison Park, PA (US); Barry L. Tucker, Verona, PA (US); Edward J. Rhinehart, Monroeville, PA (US)

(21) Appl. No.: 14/903,344

(22) PCT Filed: Jul. 10, 2014
(86) PCT No.: PCT/US14/46164

§ 371 Date: Jan. 7, 2016

Related U.S. Application Data
(60) Provisional application No. 61/845,130, filed on Jul. 11, 2013.

(51) Int. Cl. A61M 5/145

(52) U.S. Cl. CPC A61M 5/1452; A61M 2005/14533; A61M 2205/10

(57) ABSTRACT

A fluid container displacement pump, in one embodiment, includes a pump housing defining a pump chamber for holding a fluid to be dispensed to a patient, at least one actuating member movable relative to the pump housing and extending through at least one opening defined in the pump housing, and a cover member disposed in the pump chamber opposite the actuating member. The fluid container displacement pump, in another embodiment, includes a pump housing defining a pump chamber for holding a fluid to be dispensed to a patient, at least one actuating member movable relative to the pump housing and extending through at least one opening defined in the pump housing, a fluid reservoir defining a reservoir chamber in fluid communication with the pump housing, and a control valve controlling fluid flow between the reservoir chamber and the pump chamber.

### CROSS-REFERENCE TO RELATED APPLICATIONS

This application claims the benefit of U.S. Provisional Application No. 61/845,130, filed Jul. 11, 2013, the disclosure of which is hereby incorporated in its entirety by reference.

### BACKGROUND OF THE INVENTION

1. Field of the Invention

The present disclosure is directed to a fluid delivery system having a fluid pumping device for discrete volume fluid delivery applications in medical diagnostic and therapeutic procedures.

2. Description of Related Art

There are a number of medical procedures which require the delivery of fluids to a patient in a precisely controlled or discrete manner. To facilitate this requirement, a variety of fluid delivery systems have been designed and implemented. A common fluid delivery system that is used to deliver a fluid to a patient is a gravity-feed system. In this system, a bag holding the fluid is supported above the level of the patient's body and the flow rate of the fluid to the patient is monitored and controlled by the gross pressure of a clamp upon a flexible tube extending between the bag and the patient. Various manually-operated devices are also known in the medical field for delivery of fluid under pressure to a patient. In addition, a plurality of powered syringe-based infusion pumps and peristaltic pumps have also been used for delivering pressurized fluid to patients and to provide a more precise control of the volumetric delivery of the fluids.

One limitation of the syringe-based fluid injection system is the need to fill, refill and/or replace the disposable syringes prior to each patient procedure. To alleviate this problem and to provide a more precise control of volumetric delivery of fluids, positive displacement pump platforms have been developed. These devices eliminate the use of syringes and provide increased pressure ranges over peristaltic pumps. However, there are several disadvantages present in the foregoing positive displacement pump platforms known in the medical field. One such disadvantage is the lack of precision and control over the volumetric delivery of the fluid to the patient. Quite often the desired amount of fluid that is needed for the patient is very precise and, sometimes, a very small fluid volume. Current positive displacement pump platforms are often not suited to delivery of precise small fluid volumes to a patient.

### SUMMARY OF THE INVENTION

In one embodiment, a fluid container displacement pump is provided. The fluid container displacement pump includes a pump housing defining a pump chamber for holding a fluid to be dispensed to a patient, at least one actuating member movable relative to the pump housing and extending through at least one first opening defined in the pump housing, and a cover member disposed in the pump chamber opposite the at least one actuating member. Discrete movement of one or more of the at least one actuating member into the pump chamber causes a pre-determined volume of fluid in the pump chamber corresponding to the discrete movement to be dispensed from the pump chamber. Movement of the at least one actuating member in an opposing direction retracts the at least one actuating member from the pump chamber.

The cover member may include a plate disposed in the pump chamber and a shaft connected to the plate, with the shaft extending through a second opening in the pump housing. An outer surface of the shaft of the cover member may be adapted to be in operational engagement with a ratcheting member positioned externally on the pump housing. Outlet tubing may be in fluid communication with the pump chamber to deliver the pre-determined volume of fluid dispensed from the pump chamber to a patient. An outlet control valve may be provided in the outlet tubing. The outlet control valve may be configured to dispense the pre-determined volume of fluid from the pump chamber. A bladder may be disposed in the pump chamber to hold the fluid. A plate member may be connected to a distal end of the actuating member and positioned in the pump chamber. The plate member may extend across a diameter of the pump chamber. A lower surface of the cover member may define a recess shaped to receive a distal end of the at least one actuating member therein. An added weight may be provided on the cover member. An actuating device may operationally control the at least one actuating member. A second actuating device may also operationally control the cover member.

In another embodiment, a fluid container displacement pump is provided. The fluid container displacement pump includes a pump housing defining a pump chamber for holding a fluid to be dispensed to a patient, at least one actuating member movable relative to the pump housing and extending through at least one first opening defined in the pump housing, a fluid reservoir defining a reservoir chamber in fluid communication with the pump housing, and a control valve controlling fluid flow between the reservoir chamber and the pump chamber. Discrete movement of one or more of the at least one actuating member into the pump chamber causes a pre-determined volume of fluid in the pump chamber corresponding to the discrete movement to be dispensed from the pump chamber. Movement of the at least one actuating member in an opposing direction retracts the at least one actuating member from the pump chamber. Upon the pre-determined volume of fluid being dispensed from the pump chamber, fluid from the reservoir chamber enters the pump chamber under the flow control provided by the control valve.

An outlet control valve may be connected to the pump chamber to control fluid flow to outlet tubing conducting the pre-determined volume of fluid to the patient. The control valve and the outlet control valve may each comprise one-way check valves. Outlet tubing may be in fluid communication with the pump chamber to deliver the pre-determined volume of fluid dispensed from the pump chamber to a patient. A bladder may be disposed in the fluid reservoir to hold the fluid. An actuating device may operationally control the at least one actuating member. Upon a fluid pressure in the pump chamber becoming less than a fluid pressure in the reservoir chamber, fluid from the reservoir chamber may be automatically supplied to the pump chamber. The control valve may include a one-way check valve.

In another embodiment, a method of dispensing fluid from a fluid displacement pump device is provided. The method of dispensing fluid from a fluid container displacement pump includes the steps of providing the fluid displacement pumping device including a pump housing defining a pump chamber for holding the fluid to be dispensed to the patient, at least one actuating member movable relative to the pump housing and extending through at least one first opening defined in the pump housing, and a cover member disposed in the pump chamber opposite the at least one actuating member; moving one or more of the at least one actuating member into the pump chamber to pressurize the fluid; and dispensing a pre-determined volume of fluid from the pump chamber for delivery to the patient. Discrete movement of one or more of the at least one actuating member into the pump chamber causes the pre-determined volume of fluid in the pump chamber corresponding to the discrete movement to be dispensed from the pump chamber.

In another embodiment, a method of dispensing fluid from a fluid displacement pumping device is provided. The method of dispensing fluid from a fluid container displacement pump includes the steps of providing the fluid displacement pumping device including a pump housing defining a pump chamber for holding the fluid to be dispensed to the patient, at least one actuating member movable relative to the pump housing and extending through at least one first opening defined in the pump housing, a fluid reservoir defining a reservoir chamber in fluid communication with the pump housing, and a control valve controlling fluid flow between the reservoir chamber and the pump chamber; moving one or more of the at least one actuating member into the pump chamber to pressurize the fluid; and dispensing a pre-determined volume of fluid from the pump chamber for delivery to the patient. Discrete movement of one or more of the at least one actuating member into the pump chamber causes the pre-determined volume of fluid in the pump chamber corresponding to the discrete movement to be dispensed from the pump chamber.

These and other features and characteristics of the fluid displacement pumping device, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limits of the invention. As used in the specification and the claim, the singular form of “a”, “an”, and “the” include plural referents unless the context clearly dictates otherwise.

### BRIEF DESCRIPTION OF THE DRAWINGS

FIG. 1 is a perspective view of a fluid container displacement pump in accordance with one embodiment of this disclosure.

FIG. 2 is a cross-sectional view of the fluid container displacement pump in FIG. 1 along line 2-2 in FIG. 1.

FIG. 3 is a perspective view of a fluid container displacement pump in accordance with another embodiment of this disclosure.

FIG. 4 is a cross-sectional view of the fluid container displacement pump in FIG. 3 along line 4-4 in FIG. 3.

FIG. 5 is a cross-sectional view of the fluid container displacement pump shown in FIG. 1 at an exemplary displacement position.

FIG. 6 is a partial perspective and cross-sectional view of the fluid container displacement pump of FIG. 1 at an exemplary pressurized displacement position.

FIG. 7 is a cross-sectional view of the fluid container displacement pump of FIG. 1 at a return displacement position.

FIG. 8 is a cross-sectional view of the fluid container displacement pump of FIG. 1 in accordance with another embodiment of this disclosure showing a variation of the fluid container displacement pump of FIG. 1.

FIG. 9 is a cross-sectional view of a fluid container displacement pump of FIG. 1 in accordance with another embodiment of this disclosure showing a variation of the fluid container displacement pump of FIG. 1.

FIG. 10 is a cross-sectional view of a fluid container displacement pump of FIG. 1 in accordance with another embodiment of this disclosure showing a variation of the fluid container displacement pump of FIG. 1.

FIGS. 11A and 11B are sectional views of a locking arrangement for the fluid container displacement pump of FIG. 1 in accordance with one embodiment of this disclosure.

FIGS. 12A and 12B are sectional views of another locking arrangement for the fluid container displacement pump of FIG. 1 in accordance with another embodiment of this disclosure.

FIG. 13 is a sectional view showing another locking arrangement for the fluid container displacement pump of FIG. 1 in accordance with another embodiment of this disclosure.

### DESCRIPTION OF THE DISCLOSURE

For purposes of the description hereinafter, spatial orientation terms, if used, shall relate to the referenced embodiment as it is oriented in the accompanying drawing, figures, or otherwise described in the following detailed description. However, it is to be understood that the embodiments described hereinafter may assume many alternative variations and embodiments. It is also to be understood that the specific devices illustrated in the accompanying drawings, figures, and described herein are simply exemplary and should not be considered as limiting.

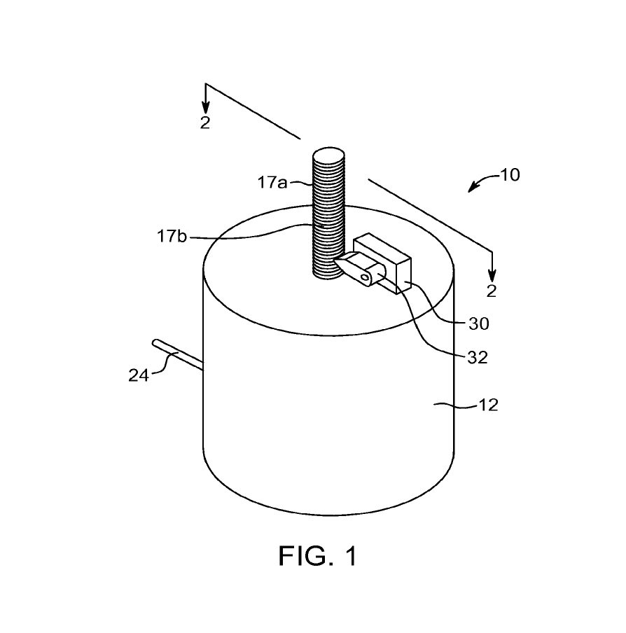

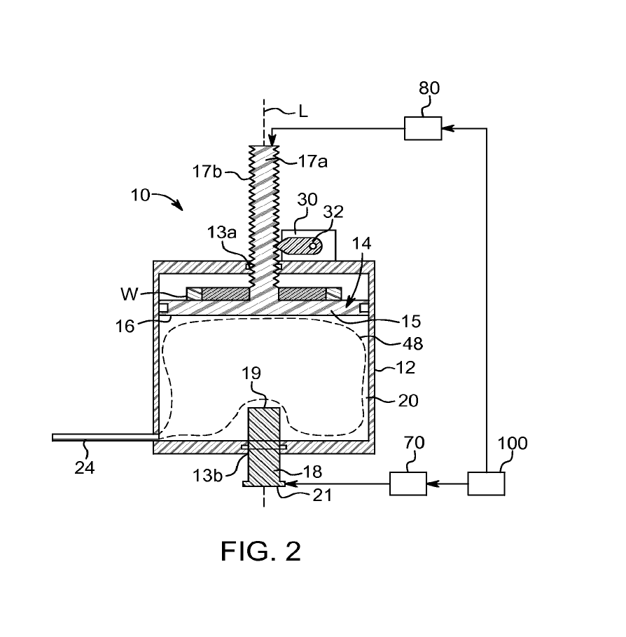

Referring to the drawings in which like reference characters refer to like parts throughout the several views thereof, a fluid container displacement pump 10 (hereinafter “pump 10”) and method for fluid delivery using the pump 10 will be described herein. Referring to FIGS. 1 and 2, the pump 10 is used as part of a fluid delivery system to deliver fluid to a patient. In this embodiment, the pump 10 generally includes a pump housing 12 and at least one actuating member 18 used to effect the positive incremental or discrete displacement of fluid from the pump 10. The following discussion initially sets forth the general structure and arrangement of the components of the pump 10, after which follows a discussion of operation of the at least one actuating member 18 to effect the positive incremental or discrete displacement of fluid from the pump 10 for several embodiments. In one embodiment, the at least one actuating member 18 may include a cylindrical rod or thin, straight bar. Other shapes, however, are contemplated for the at least one actuating member 18, including polygonal such as a generally triangular or square-shaped actuating member 18.

As noted, the pump 10 includes a housing 12 defining a pump chamber 20. The housing 12 may be a hollow, cylindrical structure made from a material suitable for medical applications, such as a medical grade plastic. Housing 12 may have other hollow shaped structures and may be made of other suitable materials, such as a metal, alloy, glass or composite. It may be desirable to construct the housing 12 from a clear medical grade plastic in order to facilitate visual verification of fluid delivery to the patient. The housing 12 serves several purposes, including as a support component or structure for a cover member 14 and the at least one actuating member 18, as well as a connection point for connecting a fluid path to a patient. The pump chamber 20 is typically filled with a fluid that is to be injected into a patient. In certain medical procedures, a precise or discrete volume of fluid, including in some embodiments, a very small volume, must be delivered to a patient, requiring a high level of precision when using a fluid pump to perform this procedure. The present embodiment of the pump 10 is able to provide this highly precise fluid volume delivery easily and efficiently. The desired fluid to be delivered to a patient is contained in the pump chamber 20. However, as shown in dashed lines in FIG. 2, the fluid may be disposed in a bladder or bag 48 placed inside of the pump chamber 20.

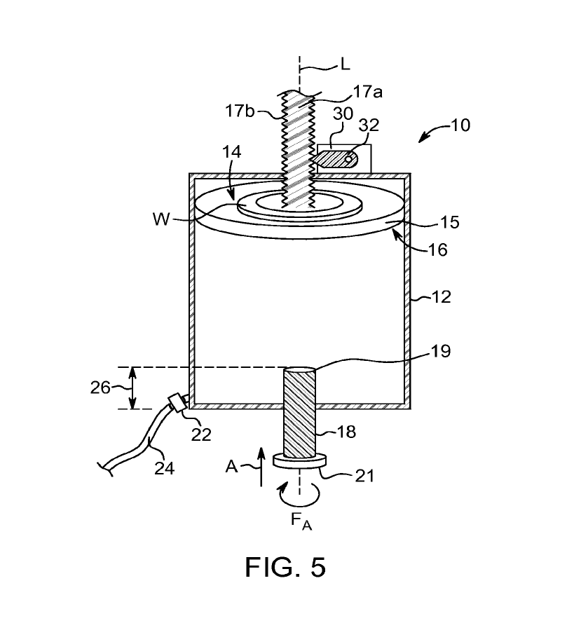

The cover member 14 is disposed within the housing 12 and includes a plate 15 positioned in contact with an inner wall of the pump chamber 20 and a shaft member 17a. A lower surface 16 of the plate 15 (as shown in FIG. 5) may be in direct contact with the fluid stored in the pump chamber 20 or in contact with an outer surface of a fluid filled bladder 48 within pump chamber 20. The shaft member 17a extends away from the plate 15 and through an opening 13a defined in the housing 12 of the pump 10. The shaft member 17a includes ratchet teeth 17b that cover at least a portion of the shaft member 17a. The ratchet teeth 17b of the shaft member 17a create a ratcheted configuration with an external ratcheting member 30 positioned on a top surface of the housing 12. The ratcheting member 30 includes a ratchet arm 32 that operatively contacts or engages the ratchet teeth 17b of the shaft member 17a. This ratcheting relationship prevents the cover member 14 from moving in an upward direction once the cover member 14 has been displaced to a lower position in the pump chamber 20. It is also to be understood that, instead of using the ratchet teeth 17b, alternative protrusions or recesses may be included on the shaft member 17a to effect a similar configuration as the ratcheted relationship between the shaft member 17a and the ratcheting member 30. Further, rotatable threads or a magnetic strip may be provided on the shaft member 17a for coupling with an encoder to measure the linear movement of the shaft member 17a. It is also contemplated that an electro-actuated clutch mechanism may be used instead of the ratchet arrangement.

The cover member 14 may be displaced along a longitudinal axis L of the pump 10 and is shaped to conform with the cross-sectional shape of the housing 12, such as the circular shape in the present embodiment, so that the cover member 14 can move within the pump chamber 20 freely and smoothly. The cover member 14 may move due to a gravitational force, by a vacuum established in the pump chamber 20, or through actuation by an actuating device 80 controlled by a controller 100. The movement of the cover member 14 may move in conjunction with a user specified delivery. In this configuration, feedback is provided to the controller 100 to report or alert the operator that the fluid was delivered to the patient. The cover member 14 may also be used as a level indicator, which allows an individual to visually or mechanically gauge the amount of fluid contained in the pump chamber 20 and/or to verify that the proper amount of fluid has been displaced from the pump chamber 20. It is also contemplated that cover member 14 may include an added weight W. The added weight may assist in providing an initial downward force on the fluid contained in the pump chamber 20. In one embodiment, the added weight W may be ring-shaped to be positioned on top of the plate 15 of the cover member 14 around the shaft member 17a as shown in FIG. 2. It is also to be understood that the added weight W may be provided in alternative configurations, such as small blocks positioned on an upper surface of the plate 15, integral with the plate 15 of the cover member 14, or elements welded, adhered, or otherwise affixed to the plate 15, among other configurations.

The at least one actuating member 18 at least partially extends into the pump chamber 20 through at least a first opening 13b defined in the pump housing 12, with an exterior portion of the at least one actuating member 18 extending outward from the pump chamber 20. The actuating member 18 is also desirably made from a material suitable for medical applications, such as medical grade plastic, glass, composite, metal or metal alloy. In the depicted embodiment of FIG. 2, a distal end 19 of the actuating member 18 is in contact with the fluid of the pump chamber 20 or in contact with an outer surface of a fluid filled bladder 48 within pump chamber 20 and a proximal end 21 is configured to engage a driving element that imparts a driving force to one or more of the at least one actuating member 18, such as a linear or rotational actuator 70, to induce movement of one or more of the at least one actuating member 18 into and out of the pump chamber 20 and apply pressure to the fluid in an upward direction or reduce pressure in the pump chamber 20 when moving in downward direction in the view of FIG. 2. The actuating member 18 may include any type of linear or rotating actuating configuration, including electrical, magnetic, hydraulic or pneumatic components. The linear or rotational actuator 70 may be in operative connection with a controller 100 that controls the actuation of each of the at least one actuating member 18. The controller 100 may actuate actuating devices 70 and 80 either simultaneously or in alternative patterns. In one embodiment, the at least one actuating member 18 is, for example, finely threaded around its outer circumferential surface. Alternatively, the at least one actuating member 18 may have one threaded portion on the proximal end 21 and another portion with a smooth surface on the distal end 19. The portion with a smooth surface may have a generally cylindrical cross-section. This smooth surface is achieved by using centerless grinding, which provides very precise tolerances and a very accurately dimensioned, smooth surface on the outer circumference of the actuating member 18. This provides a more accurate method of creating an injection device than a syringe, which requires a “draft” for molding. A syringe often includes a chamber that includes a diameter that tapers or narrows at one end of the chamber. Often a draft angle is provided in the mold for the syringe chamber to allow easier removal of the mold once the syringe chamber has been cast. This draft angle causes the syringe chamber to taper or narrow at one end. This tapering or narrowing causes inconsistent amounts of fluid to be displaced from the syringe even though an individual may apply the same amount of pressure to the syringe each time. The amount of volume that is displaced changes with every application because the volume of fluid inside of the syringe chamber is different due to the tapering or narrowing. By including a smooth portion on the distal end 19 of the at least one actuating member 18, more consistent and accurate fluid displacement amounts are achieved.

By using a finely threaded actuating member 18, an actuating device 70, whether manually operated or operated by a computer, such as a computer controlled electro-mechanical actuating device, can displace one or more of the at least one actuating member 18 in precise small increments by rotating the actuating member 18. By rotating the actuating member 18 in one direction, the actuating member 18 is displaced further into the pump chamber 20, wherein the actuating member 18 moves through a sealed opening in the pump chamber 20, which may be sealed, for example, by O-rings or other seals (not shown) located in the opening 13b in the pump chamber 20. By rotating the actuating member 18 in the opposite direction, the actuating member 18 is retracted from the pump chamber 20. With this exemplary rotational arrangement, if the actuating device rotates the at least one actuating member 18 the same rotational degree each time, the same amount or discrete volume of fluid is displaced from the pump chamber 20 with each rotation. It is also to be understood, however, that the at least one actuating member 18 may alternatively be displaced axially into and out of the pump chamber 20 by a suitable linear operator, for example a rack and pinion or cam actuating member, rather than being operated by a rotational actuating member 18. By using such a linear actuator or operator 70, the at least one actuating member 18 may be displaced into and out of the pump chamber 20 in similar precise increments.

As an actuating member 18 is rotated by an actuating device 70 such that the actuating member 18 displaces into the pump chamber 20, the actuating member 18 pressurizes the fluid in the pump chamber 20. In particular, the fluid inside of the pump chamber 20 becomes further pressurized as the distal end 19 of the actuating member 18 is displaced further into the pump chamber 20 due to the reduction in volume affected by the insertion of the actuating member 18. The pressurized fluid is conducted from the pump chamber 20 via outlet tubing 24 fluidly connected with the pump chamber 20. According to various embodiments, the pump 10 may be a closed system, wherein the only output of the closed system is to the patient through the outlet tubing 24.

It is also contemplated that the controller 100 may be operatively connected to a control interface or informatics data capture unit (not shown) configured for permitting the operator to freely and flexibly operate and identify different parameters concerning the pump 10. The control interface may display different display fields corresponding to the available quantity of fluid, a variable column of touch fields for facilitating the entry of control parameters relating to the fluid, a variable column of touch fields relating to flowrate, a variable column of touch fields relating to volume, and a display field corresponding to the available quantity of fluid. One example of such a control interface is disclosed in U.S. Pat. No. 6,643,537 to Zatezalo et al., the disclosure of which is hereby incorporated by reference in its entirety.

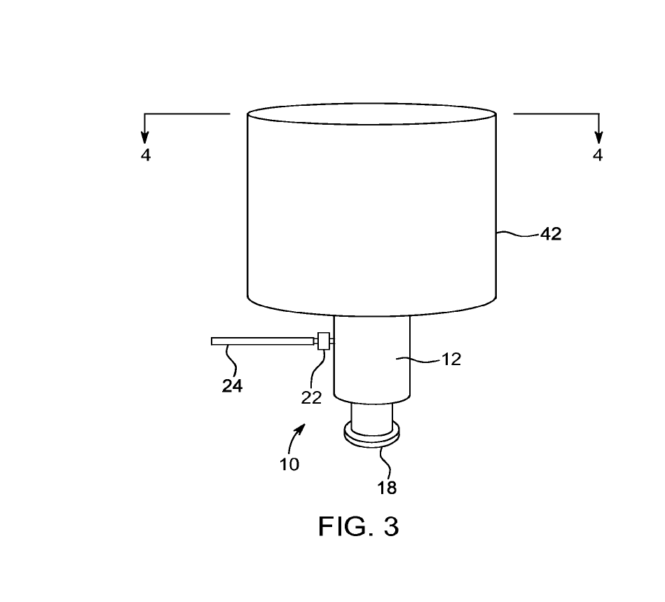

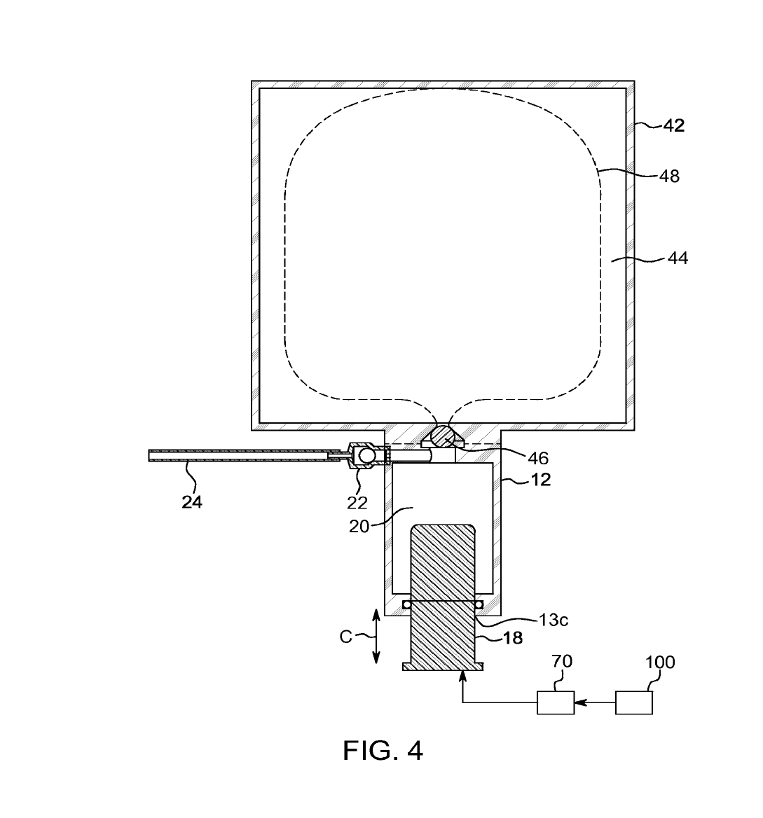

In another embodiment shown in FIGS. 3 and 4, the pump 10 may include a fluid reservoir 42 positioned, for example, above the housing 12. The fluid reservoir 42 may be provided integral with the pump housing 12 or as a separate component operatively connected to the pump housing 12. Fluid reservoir 42 may alternatively be positioned adjacent to pump 10. When provided as a separate component, the fluid reservoir 42 may be replaceable. Therefore, when all of the fluid is dispensed from one fluid reservoir 42, a new, second fluid reservoir 42 containing additional fluid may be provided in fluid communication with the pump chamber 20. This fluid reservoir 42 defines a reservoir chamber 44, which holds fluid used for injection into patients. In one embodiment, at least one bladder or bag 48 may be disposed in the reservoir chamber 44 to hold the fluid.

At least one control valve may be positioned between the reservoir chamber 44 and the outlet tubing 24. The at least one control valve may include a reservoir control valve 46 fluidly connected between the reservoir chamber 44 or reservoir bladder 48 and the pump chamber 20. In one embodiment, the reservoir control valve 46 may be a one-way check valve. Although a one-way check valve is used in this embodiment, it is contemplated that other valves, including controllable “pinch” valves, stopcocks, and automated control valves, may be used to control the flow of fluid out of the reservoir chamber 44. The reservoir control valve 46 allows fluid to flow into the pump chamber 20, but does not allow pressurized fluid from the pump chamber 20 to flow into the reservoir chamber 44. The at least one control valve may further include an outlet control valve 22 positioned in fluid communication between the pump chamber 20 and the outlet tubing 24 to control the flow of the pressurized fluid from the pump chamber 20. In one embodiment, the outlet control valve 22 is a one-way check valve. It is contemplated that other valves, including pinch valves, stopcocks, and automated control valves, may be used to control the flow of fluid out of the pump chamber 20. The outlet control valve 22 is adapted for dispensing pressurized fluid out of the pump chamber 20 and into outlet tubing 24 for injection into the patient, either directly or indirectly through a fluid delivery or control device. The outlet control valve 22 allows pressurized fluid to be dispensed from the pump chamber 20, but prevents fluid from flowing back into the pump chamber 20. This flow control feature aids in ensuring that the correct amount of fluid is injected into the patient and does not flow back into the pump chamber 20. It is also to be understood that the outlet control valve 22 may be used in various embodiments of the pump 10 as described herein, including for example, those embodiments shown in FIGS. 1 and 2. The outlet tubing 24 is desirably medical grade tubing. It is also contemplated that a similar actuating device 70 may be used to actuate the at least one actuating member 18 in this embodiment of the pump 10. The actuating device 70 may also be in communication with and controlled by a controller 100.

With reference to FIGS. 1, 2, and 5-7, operation of one embodiment of the pump 10 will now be described. This operation may be manually performed by an individual, it may be performed as an automated process, or it may be performed by a control system 100, among other types of operations which are contemplated. Although the operation of this embodiment of the pump 10 is described and shown in reference to a rotational movement of at least one of the at least one actuating member 18, it is also to be understood that the at least one actuating member 18 may be displaced axially into and axially out of the pump chamber 20. As shown in FIG. 5, as the driving force drives the actuating member 18 with a rotational force FA, the at least one actuating member 18 is displaced in an upward direction as represented by arrow A along the longitudinal axis L of the pump 10 to displacement position 26. Due to the finely threaded actuating member 18, a precise axial displacement of one or more of the at least one actuating member 18 can be calculated by, for example, a controller 100 associated with the actuating device 70 operatively connected to each of the at least one actuating member 18, with this axial displacement corresponding to a precise or discrete volume of fluid to be dispensed from the pump chamber 20 into the outlet tubing 24. The precise or discrete or pre-determined volume of fluid to be dispensed from the pump chamber 20 into the outlet tubing 24 corresponds to discrete or incremental rotational movement of one or more of the at least one actuating member 18. As the actuating member 18 is displaced further into the pump chamber 20, the fluid becomes further pressurized.

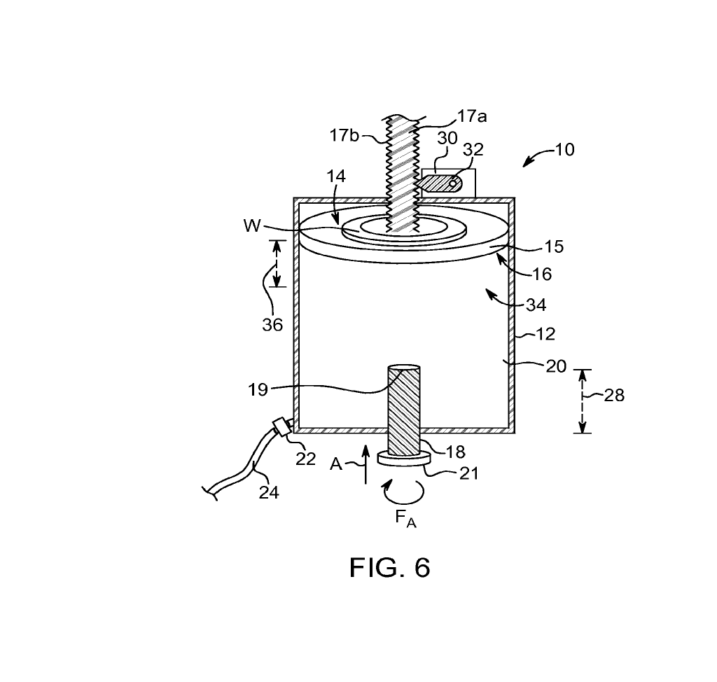

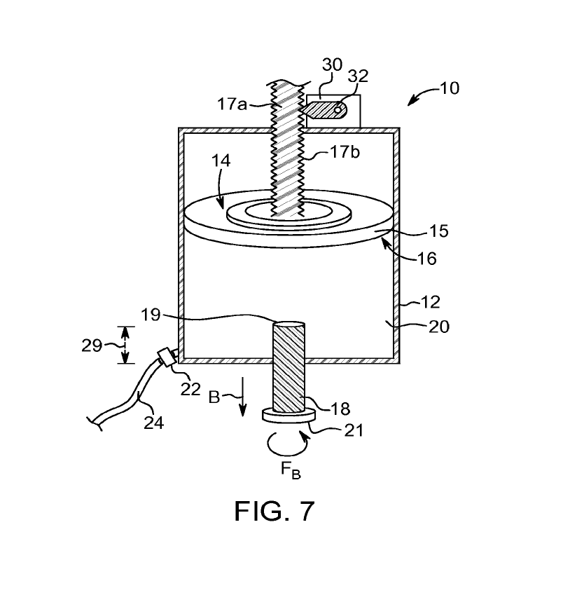

Once the fluid is sufficiently pressurized, such as when the actuating member 18 is in a desired pressurized displacement position 28 (shown in FIG. 6), the fluid may be dispensed from the pump chamber 20 through the outlet tubing 24. In the pressurized displacement position 28, the at least one actuating member 18 is axially closer to the cover member 14 than when the actuating member 18 is positioned in the initial displacement position 26 shown in FIG. 5. Once in the desired fluid volume has been delivered to the patient, the at least one actuating member 18 may be retracted in axial direction in the pump chamber 20. As the actuating member 18 is retracted from the pump chamber 20 in a direction represented by arrow B (FIG. 7), a vacuum 36 is created in the pump chamber 20 due to the reduced volume of fluid in the pump chamber 20. When using a bladder or bag 48, as illustrated in FIG. 2, the vacuum 36 is not created, rather the bladder or bag 48 is collapsed as the bladder or bag 48 is emptied. A vacuum, therefore, is not developed or created in the bladder or bag 48.

Once a desired volume of fluid has been dispensed from the pump chamber 20, the actuating device may apply a rotational force Fs to the at least one actuating member 18 to return the actuating member to a return displacement position 29 (shown in FIG. 7). Once the return displacement position 29, the at least one actuating member 18 can be actuated by applying a rotational force F_{A} again to provide another dose of fluid to the patient. As the actuating member 18 is retracted from the pump chamber 20, the cover member 14 may also be actuated by an actuating device 80 to move along the longitudinal axis L of the pump 10 towards the actuating member 18 to remove the vacuum space or condition 36 created by the dispensed fluid volume. Alternatively, the cover member 14 may move along the longitudinal axis L of the pump 10 towards the actuating member 18 due to gravitational force on the weight of the cover member 14 and/or the added weight W. The cover member 14 is displaced to a reduced fluid volume level 34 in the pump chamber 20 to remove the vacuum space 36. The at least one actuating member 18 may then be displaced into the pump chamber 20 to once again to pressurize the fluid therein. When the actuating member 18 is moved to the return displacement position 29, the actuating member 18 is a shorter distance away from the cover member 14 than when the actuating member 18 is positioned in the initial displacement position 26 because the cover member 14 has been displaced further into the pump chamber 20. This operation can be repeatedly performed by a user or an actuating device to inject a desired amount of fluid to a patient or patients by displacing the at least one actuating member 18 into the pump chamber 20 again. The desired amounts of fluid delivered to the patient(s) may be adjusted in each operation by adjusting the displacement position of the actuating member 18 within the pump chamber 20. Although the foregoing method of operating the pump 10 was described with reference to one specific embodiment of the pump 10, it is to be understood that the pump 10 may also operate with the bladder or bag 48 shown in FIGS. 2 and 8-10.

The pump 10 provides an efficient and effective way to inject precise volumes of fluid into a patient or into multiple patients. By including fine threads on the outer circumference of the at least one actuating member 18, a user or actuating device can displace one or more of the at least one actuating member 18 in small, accurate increments into the pump chamber 20. These small increments in axial displacement correlate to small fluid volumes that are displaced from the pump chamber 20. In one embodiment, the at least one actuating member 18 can be displaced into the pump chamber 20 so as to accurately dispense from 0.1 ml to 200 ml of fluid from the pump chamber 20. This precise control of the volume of fluid to be injected into the patient can be useful in situations where the fluid volume to be injected is important or desirable to the effective treatment of the patient. The operation of the pump 10 also allows for the quick injection of the proper or desired volume of fluid into the patient because it is known in advance how many turns of the actuating member 18 are required to provide the proper volume of fluid for injection.

It is also to be understood that alternative methods of measuring the fluid injection volume may be used. For example, instead of fine threading on the actuating member 18, the at least one actuating member 18 may include protrusions or recesses that correspond to protrusions or recesses on the pump housing 12. In this alternative embodiment, each protrusion corresponds to a specific volume fluid that may be displaced from the pump 10, similar to the fine threading of the actuating member 18. It is also contemplated that a linear or rotational actuator 70 may be programmed to exert a predetermined force on the actuating member 18 that corresponds to a predetermined distance that the at least one actuating member 18 to be inserted into the pump chamber 20. It is also to be understood that, although the actuating devices 70 and 80 and the controller 100 are not shown in FIGS. 5-7, the actuating devices 70 and 80 and the controller 100 may be provided to actuate the at least one actuating member 18 and the cover member 14.

As shown in FIGS. 5-7, the pump 10 may also operate using an outlet control valve 22. In one embodiment of the operation of the pump 10, the outlet control valve 22 may be a high pressure check valve. In this configuration, the fluid contained in the pump chamber 20 is set at an initial pressure. As one or more of the at least one actuating member 18 is moved further into the pump chamber 20, the pressure of the fluid in the pump chamber 20 is primed to a value slightly less than the pressure threshold of the outlet control valve 22. After the internal pressure of the pump chamber 20 has been primed, further movement of the at least one actuating member 18 into the pump chamber 20 will increase the pressure of the fluid to be greater than the pressure threshold of the outlet control valve 22. The fluid is then discharged from the pump chamber 20 and delivered to the patient via the outlet tubing 24.

Referring to FIG. 4, operation of the depicted embodiment of the pump 10 will now be generally described. This embodiment includes the fluid reservoir 42, the outlet control valve 22, and the reservoir control valve 46. As with the previous embodiment, an individual or actuating device 70 actuates the at least one actuating member 18 by axially displacing or rotating the at least one actuating member 18 to cause movement in upward direction represented by arrow C. As the at least one actuating member 18 is displaced into the pump chamber 20, the fluid becomes pressurized. Once the fluid is sufficiently pressurized, the outlet control valve 22 is opened. The pressurized fluid is dispensed through the outlet control valve 22 and the outlet tubing 24 to a patient. During operation, a pressure differential is developed between the pump chamber 20 and the reservoir chamber 44. As the at least one actuating member 18 is withdrawn from the pump chamber 20, an at least partial vacuum is created inside of the pump chamber 20. By reducing the pressure in the pump chamber 20, the fluid pressure in the pump chamber 20 becomes lower than the head pressure in the reservoir chamber 44. Due to this difference in pressure, fluid contained in the reservoir chamber 44 opens the reservoir control valve 46 and is dispensed through the reservoir control valve 46 into the pump chamber 20. This reservoir fluid flows into the pump chamber 20 and fills the at least partial vacuum that has been created from the actuating member 18 retraction, thereby keeping the pump chamber 20 at a desired volume at all times. The discrete or incremental movement of one or more of the at least one actuating member 18 into the pump chamber 20 again correlates or corresponds to the pre-determined volume of fluid to be delivered to the patient in the same manner as the embodiment shown in FIG. 2.

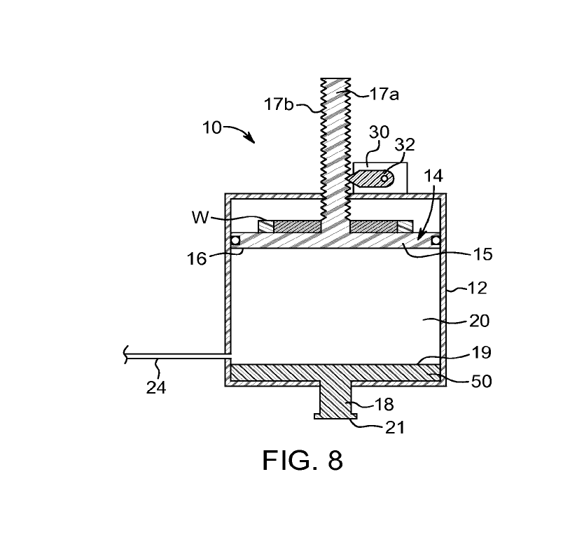

Referring to FIG. 8, another embodiment of the pump 10 is shown. The pump 10 is substantially similar to the pump 10 shown in FIGS. 1 and 2. However, the actuating member 18 includes an additional feature and the outlet tubing 24 is positioned at a different location on the pump housing 12. In this embodiment, the actuating member 18 includes a plate member 50. The plate member 50 is provided on the distal end 19 of the actuating member 18. The plate member 50 generally corresponds to the inner diameter and cross-sectional shape of the pump chamber 20. It is contemplated, however, that the plate member 50 may have a different diameter or cross-sectional shape than the pump chamber 20.

As the actuating member 18 is actuated, the plate member 50 moves into the pump chamber 20 towards the cover member 14. As the actuating member 18 is moved in an opposite direction, the plate member 50 is returned to its original position adjacent the bottom surface of the pump chamber 20. In this embodiment of the pump 10, the outlet tubing 24 is positioned at a higher level on the pump housing 12 than the outlet tubing 24 shown in FIG. 2. The location of the outlet tubing 24 in FIG. 8 corresponds approximately to the height of the plate member 50 relative to the pump housing 12. With this configuration, the surface of the pump chamber 20 that the fluid rests on, which effectively correlates to the top surface of the plate member 50, remains level with the outlet tubing 24. The plate member 50 is configured to maximize the volume of fluid that can be dispensed from the pump 10. In this embodiment, nearly all of the fluid contained in the pump 10 may be dispensed via the outlet tubing 24. The plate member 50 prevents fluid from settling in dead spaces around the actuating member 18 on the bottom surface of the pump chamber 20, as can occur with the at least one actuating member 18 of FIG. 2. In the embodiment illustrated in FIG. 8, the plate member 50 is configured to direct all of the fluid out of the pump 10 since the plate member 50 extends across the entire diameter of the pump chamber 20.

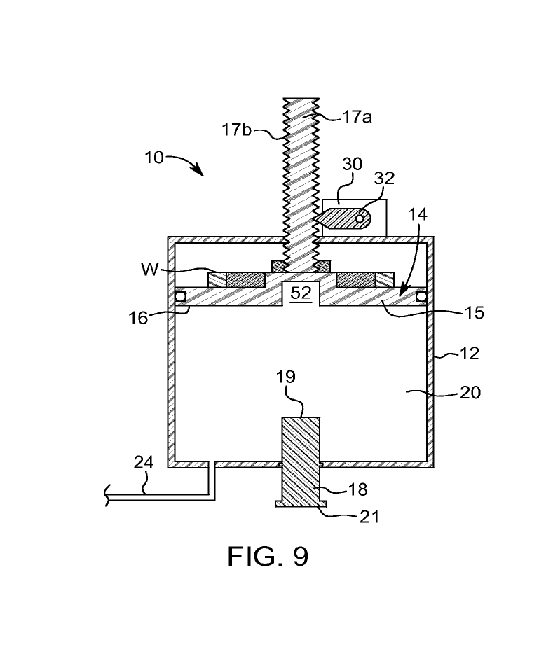

Referring to FIG. 9, another embodiment of the pump 10 is shown. The pump is substantially similar to the pump 10 shown in FIG. 2. However, the cover member 14 includes at least one hole or aperture or recess 52 defined in a lower surface 16 of the plate 15 configured to receive the distal end 19 of the at least one actuating member 18. As fluid is displaced from the pump 10, the cover member 14 is lowered towards the bottom surface of the pump chamber 20 to fill the vacuum space 36 created by the dispensed fluid. As the cover member 14 is moved lower relative to the pump chamber 20, the cover member 14 eventually comes into contact with the distal end 19 of the at least one actuating member 18. By providing an at least one aperture or recess 52 in the plate 15 of the cover member 14, the plate 15 may receive the distal end 19 of the at least one actuating member 18 in the at least one aperture or recess 52 and move further downward in the pump chamber 20 to contact the bottom surface of the pump chamber 20. With this configuration, nearly all of the fluid contained in the pump chamber 20 may be dispensed from the pump 10 via the outlet tubing 24. The outlet tubing 24 is provided in a bottom surface of the pump chamber 20 to allow all of the fluid to discharge from the pump chamber 20. According to this embodiment, fluid is not left in dead spaces located below the distal end 19 of the actuating member 18 due to the distal end 19 of the at least one actuating member 18 impeding the movement of the plate 15 of the cover member 14.

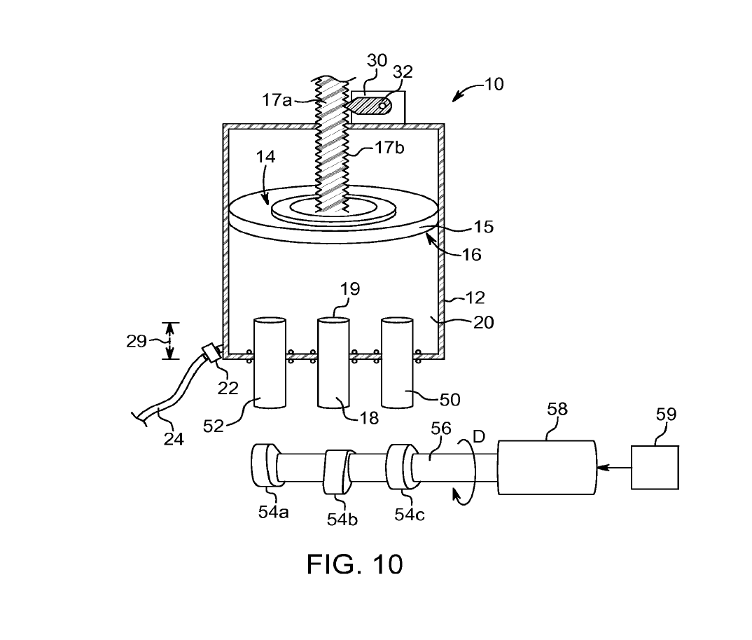

Referring to FIG. 10, another embodiment of the pump 10 is shown. The pump 10 is substantially similar to the pump 10 shown in FIG. 2. However, additional multiple actuating members 50, 52 are provided to discharge a larger volume of fluid from the pump chamber 20 compared to a single actuating member 18. Similar to the actuating member 18, the additional actuating members 50, 52 extend through a bottom surface of the pump housing 12. The additional actuating members 50, 52 may also be substantially cylindrical or have a shape similar to or different from the actuating member 18. The additional actuating members 50, 52 are slidably disposed in the pump housing 12 and may be inserted and withdrawn from the pump chamber 20 in a similar fashion to the actuating member 18. In one embodiment, the actuating members 18, 50, 52 may be driven by a cam drive arrangement. The cam drive arrangement may include at least one cam lobe 54a, 54b, 54c that corresponds to each actuating member 18, 50, 52. The cam lobes 54a, 54b, 54c are positioned on a cam shaft 56 that is driven in a rotational direction D by a rotational actuator 58. As the actuator 58 rotates the cam shaft 56, the cam lobes 54a, 54b, 54c are rotated to come in contact with the respective actuating members 18, 50, 52. The cam lobes 54a, 54b, 54c push the actuating members 18, 50, 52 into the pump chamber 20 to discharge fluid from the pump chamber 20 to a patient via the outlet tubing 24 in a similar fashion as described herein. By providing additional actuating members 50, 52, more fluid is provided to the patient for operations or procedures that require a larger volume of fluid. Further, the cam lobes 54a, 54b, 54c may be angularly offset from one another on the cam shaft 56 so as to be positioned out of phase with one another. Using this configuration, the fluid may be continuously discharged from the pump chamber 20. As the actuating member 50 is contacted by the cam lobe 54c and pushed into the pump chamber 20, fluid is discharged via the outlet tubing 24. Since the next cam lobe 54b is offset from the first cam lobe 54c, shortly after the cam lobe 54c contacts the actuating member 50, the next cam lobe 54b contacts the next actuating member 18 to push the actuating member 18 into the pump chamber 20 to discharge fluid from the pump chamber 20. Likewise, the last cam lobe 54a pushes the last actuating member 52 into the pump chamber 20 shortly after the actuating member 18 is pushed into the pump chamber 20. This process may be repeated to provide a continuous supply of fluid to a patient via the outlet tubing 24. Further, according to certain embodiments, fluctuations or pulsitility of the fluid flow to the patient may be reduced due to the features of the offset cam lobe set up. The actuator 58 may be in operative connection with a controller 59 that controls the actuation of the actuator 58. Other embodiments may comprise alternative methods for actuating the actuating members 18, 50, and 52, such as a threading on the outer circumference of the actuating members along with a rotational actuator that rotates actuating members 18, 50, and 52 to drive them into or out of the chamber 20, either together or in an alternating arrangement.

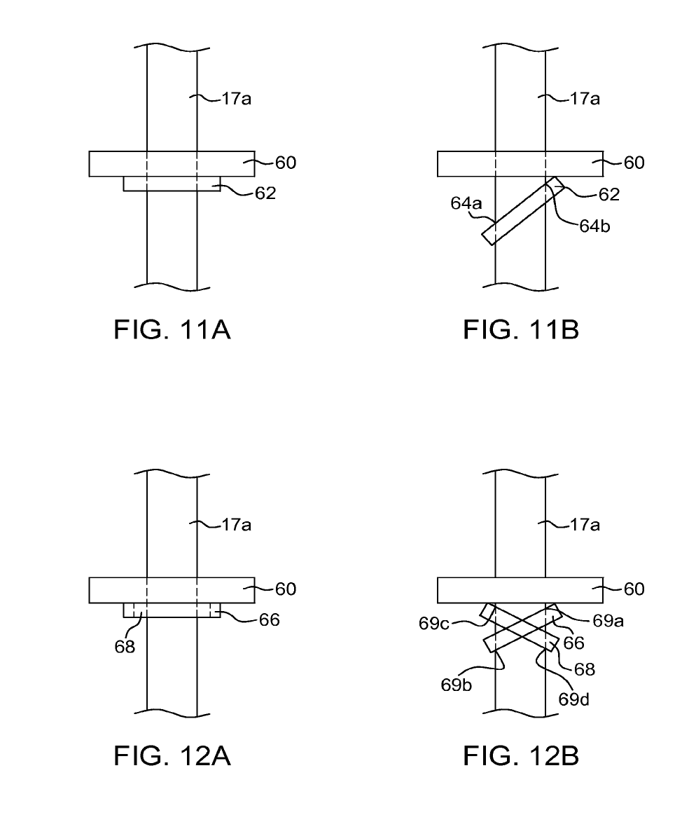

Referring to FIGS. 11A-13, alternative embodiments of the fluid container displacement pump are shown. With specific reference to FIGS. 11A and 11B, a connection interface for permitting the shaft member 17a to move downwardly into the pump chamber 20 is shown in accordance with one embodiment. The connection interface includes at least one shaft retaining plate 60 provided on the shaft member 17a and a corresponding locking plate 62 provided on the shaft member 17a. For example, the at least one shaft retaining plate 60 and the corresponding locking plate 62 may be provided on an outer surface of the shaft member 17a. In certain embodiments, the locking plate 62 may be hingably affixed to shaft retaining plate 60 to allow pivoting of retaining plate 60 relative to locking plate 62.

With continuing reference to FIGS. 11A and 11B, the locking plate 62 is configured to operatively engage the shaft retaining plate 60. The shaft retaining plate 60 and the locking plate 62 are configured to receive the shaft member 17a therethrough. The locking plate 62 is positioned below the shaft retaining plate 60 and angled relative to the shaft member 17a. While FIGS. 11A and 11B show the locking plate 62 positioned below the shaft retaining plate 60, the locking plate 62 may also be positioned above the shaft retaining plate 60. It is also contemplated that a locking plate 62 may be positioned above the shaft retaining plate 60 and a locking plate 62 may be positioned below the shaft retaining plate 60.

In a first state, such as before the shaft member 17a has moved downwardly in the pump chamber 20, the locking plate 62 is angled relative to the shaft member 17a. In this angled position, the locking plate 62 contacts the shaft member 17a at two separate points 64a, 64b. The contact points 64a, 64b restrict the movement of the shaft member 17a relative to the shaft retaining plate 60 and the pump chamber 20. As the locking plate 62 is rotated relative to the shaft member 17a, the contact points 64a, 64b are removed. Continued movement of the locking plate 62 aligns the locking plate 62 with the shaft retaining plate 60. Once the locking plate 62 and the shaft retaining plate 60 are aligned with one another (as shown in FIG. 11A), the shaft member 17a may move upwardly and downwardly relative to the shaft retaining plate 60 and the locking plate 62. The locking plate 62 may be moved by any type of electrical, magnetic, hydraulic, or electromechanical actuator. It is also contemplated that a controller (not shown) may move the locking plate 62 in unison with the movement of the actuating member 18 so that the shaft member 17a moves downwardly in the pump chamber 20 as the actuating member 18 discharges fluid from the pump chamber 20.

Referring to FIGS. 12A and 12B, at least two locking plates 66, 68 may be provided on an outer surface of the shaft member 17a, along with a shaft retaining plate 60. One locking plate 68 is provided within an aperture defined by a second locking plate 66. The locking plates 66, 68 operate in a similar fashion to the locking plate 62 of FIGS. 11A and 11B, but create four contact points 69a-69d with shaft member 17a instead of two contact points.

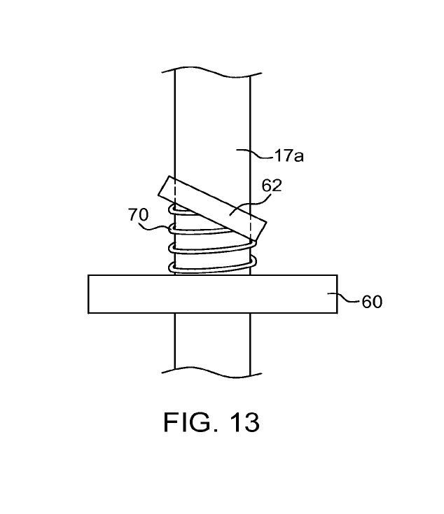

Referring to FIG. 13, a spring 70 is provided on the shaft member 17a such that the spring 70 is disposed between the locking plate 62 and the shaft retaining plate 60. The spring 70 is deflectable and provides a resilient restoring force when the locking plate 62 is urged in a downward direction towards the shaft retaining plate 60. In a first state, the spring 70 urges the locking plate 62 away from the shaft retaining plate 60 such that the locking plate 62 is angled relative to the shaft retaining plate 60. As the locking plate 62 is moved downwardly towards the shaft retaining plate 60, for example, as the shaft member 17b is moved downward, the spring 70 is compressed. The compression of the spring 70 provides a restoring force against the locking plate 62. As pressure on the locking plate 62 is decreased, the restoring force pushes the locking plate 62 back into an angled position relative to the shaft retaining plate 60, thereby locking the shaft member 17a. Use of the various embodiments of shaft retaining plate 60 and locking plate 62 allow control of the movement of the shaft member 17b during operation of pump 10.

While several embodiments of a fluid container displacement pump are shown in the accompanying figures and described hereinabove in detail, other embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A fluid container displacement pump, comprising:
a pump housing defining a pump chamber for holding a fluid to be dispensed to a patient;
at least one actuating member movable relative to the pump housing and extending through at least a first opening defined in the pump housing; and
a cover member disposed in the pump chamber opposite the at least one actuating member,
wherein discrete movement of one or more of the at least one actuating member into the pump chamber causes a pre-determined volume of the fluid in the pump chamber corresponding to the discrete movement to be dispensed from the pump chamber, and movement of the at least one actuating member in an opposing direction retracts the at least one actuating member from the pump chamber.

2. The fluid container displacement pump as claimed in claim 1, wherein the cover member comprises a plate disposed in the pump chamber and a shaft connected to the plate, wherein the shaft extends through a second opening in the pump housing, and wherein an outer surface of the shaft of the cover member is adapted for operational engagement with a ratcheting member positioned externally on the pump housing.

3. The fluid container displacement pump as claimed in claim 1, further comprising outlet tubing in fluid communication with the pump chamber to deliver the pre-determined volume of the fluid dispensed from the pump chamber to a patient.

4. The fluid container displacement pump as claimed in claim 3, further comprising an outlet control valve in the outlet tubing, wherein the outlet control valve is configured to dispense the pre-determined volume of the fluid from the pump chamber to the patient.

5. The fluid container displacement pump as claimed in claim 1, further comprising a bladder disposed in the pump chamber to hold the fluid.

6. The fluid container displacement pump as claimed in claim 1, further comprising a plate member connected to a distal end of the at least one actuating member and positioned in the pump chamber.

7. The fluid container displacement pump as claimed in claim 1, wherein a lower surface of the cover member defines at least one recess shaped to receive a distal end of the at least one actuating member therein.

8. The fluid container displacement pump as claimed in claim 1, further comprising an added weight provided on the cover member.

9. The fluid container displacement pump as claimed in claim 1, further comprising an actuating device operationally controlling the at least one actuating member.

10. The fluid container displacement pump as claimed in claim 1, further comprising a second actuating device operationally controlling the cover member.

11. A fluid container displacement pump, comprising:
a pump housing defining a pump chamber for holding a fluid to be dispensed to a patient;
at least one actuating member movable relative to the pump housing and extending through at least a first opening defined in the pump housing;
a fluid reservoir defining a reservoir chamber for holding additional fluid and in fluid communication with the pump housing; and
a control valve controlling fluid flow between the reservoir chamber and the pump chamber,
wherein discrete movement of one or more of the at least one actuating member into the pump chamber causes a pre-determined volume of the fluid in the pump chamber corresponding to the discrete movement to be dispensed from the pump chamber, and movement of the at least one actuating member in an opposing direction retracts the at least one actuating member from the pump chamber, and
wherein, upon the pre-determined volume of the fluid being dispensed from the pump chamber, the additional fluid from the reservoir chamber enters the pump chamber under the flow control provided by the control valve.

12. The fluid container displacement pump as claimed in claim 11, further comprising an outlet control valve connected to the pump chamber to control fluid flow to outlet tubing conducting the pre-determined volume of the fluid to the patient.

13. The fluid container displacement pump as claimed in claim 12, wherein the control valve and the outlet control valve each comprise one-way check valves.

14. The fluid container displacement pump as claimed in claim 11, further comprising outlet tubing in fluid communication with the pump chamber to deliver the pre-determined volume of the fluid dispensed from the pump chamber to the patient.

15. The fluid container displacement pump as claimed in claim 11, further comprising a bladder disposed in the fluid reservoir to hold the additional fluid.

16. The fluid container displacement pump as claimed in claim 11, further comprising an actuating device operationally controlling the at least one actuating member.

17. The fluid container displacement pump as claimed in claim 11, wherein, upon a fluid pressure in the pump chamber becoming less than a fluid pressure in the reservoir chamber, the additional fluid from the reservoir chamber is automatically supplied to the pump chamber.

18. The fluid container displacement pump as claimed in claim 11, wherein the control valve comprises a one-way check valve.

19. A method of dispensing a fluid from a fluid container displacement pump, comprising:
providing the fluid container displacement pump, comprising:
a pump housing defining a pump chamber for holding the fluid to be dispensed to a patient;
at least one actuating member movable relative to the pump housing and extending through at least a first opening defined in the pump housing; and
a cover member disposed in the pump chamber opposite the at least one actuating member,
moving one or more of the at least one actuating member into the pump chamber to pressurize the fluid; and
dispensing a pre-determined volume of the fluid from the pump chamber for delivery to the patient,
wherein discrete movement of the one or more of the at least one actuating member into the pump chamber causes the pre-determined volume of the fluid in the pump chamber corresponding to the discrete movement to be dispensed from the pump chamber.

20. A method of dispensing a fluid from a fluid container displacement pump, comprising:
providing the fluid container displacement pump, comprising:
a pump housing defining a pump chamber for holding the fluid to be dispensed to a patient;
at least one actuating member movable relative to the pump housing and extending through at least a first opening defined in the pump housing;
a fluid reservoir defining a reservoir chamber for holding additional fluid and in fluid communication with the pump housing; and
a control valve controlling fluid flow between the reservoir chamber and the pump chamber,
moving one or more of the at least one actuating member into the pump chamber to pressurize the fluid; and
dispensing a pre-determined volume of the fluid from the pump chamber for delivery to the patient,
wherein discrete movement of the one or more of the at least one actuating member into the pump chamber causes the pre-determined volume of the fluid in the pump chamber corresponding to the discrete movement to be dispensed from the pump chamber.
